(19) United States
(12) Patent Application Publication
(10) Pub. No.: US 2013/0197326 A1
(43) Pub. Date: Aug. 1, 2013

(54) Compositions And Methods For Measurement of Analytes

(71) Applicants: John Matthew DUBACH, Somerville, MA (US); Heather A. CLARK, Lexington, MA (US); Karen K. GLEASON, Cambridge, MA (US); Gozde OZAYDIN-INCE, Kadikoy Instanbul (TR)

(72) Inventors: John Matthew DUBACH, Somerville, MA (US); Heather A. CLARK, Lexington, MA (US); Karen K. GLEASON, Cambridge, MA (US); Gozde OZAYDIN-INCE, Kadikoy Instanbul (TR)

(73) Assignee: NORTHEASTERN UNIVERSITY, Boston, MA (US)

(21) Appl. No.: 13/608,942

(22) Filed: Sep. 10, 2012

Related U.S. Application Data
(63) Continuation of application No. 13/360,307, filed on Jan. 27, 2012, now abandoned.

(51) Int. Cl. A61B 5/1459; C09K 11/06; A61B 5/145; G01N 33/53

(52) U.S. Cl. CPC A61B 5/1459; G01N 33/5306; C09K 11/06; A61B 5/14546; A61B 5/14532
USPC 600/314; 422/69; 264/255; 264/21; 600/317; 600/316; 600/321

(57) ABSTRACT

Disclosed herein are compositions comprising an oblong optode sensing agent. The oblong optode sensing agent comprises a core and a semipermeable membrane, wherein the core comprises one or more sensors configured to bind to an analyte. In addition, methods of making and detecting the oblong optode sensing agents are disclosed.

### CROSS-REFERENCE TO RELATED APPLICATIONS

This application is a continuation of U.S. application Ser. No. 13/360,307, filed Jan. 27, 2012, entitled Compositions And Methods For Measurement Of Analytes, the contents of which are incorporated herein.

### STATEMENT REGARDING FEDERALLY SPONSORED RE SEARCH/DEVELOPMENT

This invention was made with government support under Grant No. W911NF-07-D-0004 awarded by Army Research Office. In addition, the invention was made with government support under DGE 0504331 awarded by National Science Foundation. The government has certain rights in the invention.

### FIELD OF THE INVENTION

The invention is generally directed to compositions and methods for measuring analytes in vivo and in vitro.

### BACKGROUND OF THE INVENTION

Analyte detection is an important component in biotechnology, analytical chemistry, analysis of environmental samples, and medical diagnostics. Certain types of detection assays, such as fluorescence-based assays, are capable of providing detailed pictures of where fluorescent molecules are localized in tissues and cells. In particular, fluorescence-based assays exhibit exceptional sensitivity, detecting small concentrations of fluorescent molecules.

In addition, direct, minimally invasive monitoring of in vivo physiological conditions presents a route to determine health status in real time and address needs as they arise. Continuously monitoring sodium in vivo addresses multiple diseases and could prevent clinical complications during certain procedures. Sodium imbalances may lead to hypernatremia (Adrogue H J, Madias N E (2000) N Engl J Med 342:1483-1499) or hyponatremia (Adrogue H J, Madias N E (2000) N Engl J Med 342:1581-1589)—the most common electrolyte disorder. Monitoring sodium may provide an insight into the progression of subarachnoid hemorrhage or syndrome of inappropriate antidiuresis (Benvenga S (2006) Nat Clin Pract Endocrinol Metab 2:608-609; Ellison D H, Berl T (2007) N Engl J Med 356:2064-2072). However, to provide continuous monitoring, sensors need to be small enough to have a rapid response to changes in concentration yet be large enough to reside at the sight of administration without diffusing away or being endocytosed/phagocytosed by cells.

However, the only commercially available methods to measure analytes are through blood withdrawals and detection through either a glucometer or ELISA detection kit. These approaches require the removal of blood and can only be made when blood is drawn. Furthermore, presently available approaches utilize sensors that are insufficient for in vivo monitoring of analytes because the sensors do not remain at the site of administration and therefore do not provide an opportunity to detect the sensors.

Therefore, there is a need for compositions and methods for the inexpensive and rapid assaying biological, environmental, and chemical samples.

### SUMMARY OF THE INVENTION

According to aspects of the present disclosure, sensing agents having a shape that allows for sustained localization of optode sensing agents at a site of in vivo administration of the sensing agents in a tissue. The optode sensing agents have a particular shape that provides a high surface-to-volume ratio that allows for accurate measurement of analytes and for reduced dispersion of nanoparticles in the tissue of administration. Aspects of the invention also involve methods of measuring the fluorescence in a sample utilizing the nanoparticles disclosed herein. Furthermore, the compositions and methods disclosed herein provide for rapid and simple measurement of analytes in tissues, such as epithelial and endothelial tissues.

Aspects disclosed herein provide an oblong optode sensing agent. The oblong optode sensing agent comprises a core and a semipermeable membrane, wherein the core comprises one or more sensors configured to bind to an analyte. In certain embodiments, the one or more sensors covalently bind to the analyte. In particular embodiments, the analyte is selected from the group consisting of electrolytes, salts, hormones, steroids, small molecules, drugs, and saccharides. In other embodiments, the one or more sensors are fluorescent sensors.

In some embodiments, the core further comprises a polymer. In certain embodiments, the core further comprises a plasticizer. In other embodiments, the semipermeable membrane comprises a hydrogel. In more embodiments, the semipermeable membrane comprises a biocompatible hydrogel. In further embodiments, the semipermeable membrane is permeable to analyte and impermeable to the one or more sensors.

In still further embodiments, the core comprises one or more polymers selected from the group consisting of polyvinyl chloride, polylactic co-glycolic acid, methacrylate, and polycaprolactone.

In still other embodiments, the optode sensing agent has a circular cross-section. In particular embodiments, the optode sensing agent has a rectangular shape. In more particular embodiments, the optode sensing agent has a length of about 40 μm to about 60 μm.

In certain embodiments, the optode sensing agent has a diameter of from about 200 nm to about 500 nm. In other embodiments, the optode sensing agent has a length of about 40 μm to about 60 μm. In more embodiments, the optode sensing agent has a width of from about 200 nm to about 500 nm.

In further embodiments, the semipermeable membrane comprises a confused surface. In other embodiments, the semipermeable membrane comprises poly(2-hydroxyethyl methacrylate).

In particular embodiments, the sensor is soluble in an organic solvent.

In some embodiments, the core has a diameter of less than or equal to about 100 nm. In other embodiments, the semipermeable membrane has a thickness of about 50 nm.

Aspects of the methods disclosed herein provide methods of making oblong optode sensing agents. The methods comprise providing a mold comprising one or more pores and coating the interior surfaces of the one or more pores with a first material, the first material forming a semipermeable membrane. The methods further entail applying a second material to the one or more coated pores from b) such that the second material fills the one or more coated pores, the second material comprising one or more sensors and applying the first material to the ends of the one or more coated pores filled with the second material. The methods also comprise permitting the first and second materials to form the oblong optode sensing agents and releasing the oblong optode sensing agents from the mold.

In certain embodiments, the first material is a biocompatible hydrogel. In other embodiments, the first material is poly(2-hydroxyethyl methacrylate). In still other embodiments, the second material comprises a polymer. In further embodiments, the polymer is selected from the group consisting of polyvinyl chloride, polylactic co-glycolic acid, methacrylate, and polycaprolactone

In still further embodiments, the second material further comprises a plasticizer.

In particular embodiments, the one or more pores have a width of about 200 nm. In more particular embodiments, the mold is an aluminum oxide scaffold. In other particular embodiments, releasing the oblong optode sensing agents from the mold further comprises etching the mold with an acid or a base. In certain embodiments, releasing the oblong optode sensing agents further comprises sonication of the oblong optode sensing agents.

In still other embodiments, the one or more sensors are fluorescent sensors.

Additional aspects disclosed herein relate to methods of detecting an analyte. In certain aspects, the analyte is detected in a tissue of a subject. In these aspects, the methods comprise implanting a plurality of oblong optode sensing agents in the tissue. Each oblong sensing agent comprises: i) a core having one or more fluorescent sensors configured to bind to the analyte, and ii) a semipermeable membrane. Furthermore, the methods comprise contacting the plurality of oblong sensing agents with the analyte and detecting the analyte in the tissue.

In certain embodiments, the analyte is selected from the group consisting of electrolytes, salts, hormones, steroids, small molecules, drugs, and saccharides. In other embodiments, the core further comprises a polymer. In other embodiments, the semipermeable membrane comprises a hydrogel. In particular embodiments, the semipermeable membrane comprises a biocompatible hydrogel. In more particular embodiments, the semipermeable membrane is permeable to the analyte and impermeable to the one or more fluorescent sensors.

In certain embodiments, the core comprises one or more polymers selected from the group consisting of polyvinyl chloride, polylactic co-glycolic acid, methacrylate, and polycaprolactone

In other embodiments, the plurality of oblong optode sensing agents has a circular cross-section. In still other embodiments, the plurality of oblong optode sensing agents has a rectangular shape. In further embodiments, the plurality of oblong optode sensing agents has a length of about 40 μm to about 60 μm. In still further embodiments, the plurality of oblong optode sensing agents has a diameter of from about 200 nm to about 500 nm. In still more embodiments, the plurality of oblong optode sensing agents has a length of about 40 μm to about 60 μm. In yet more embodiments, the plurality of optode sensing agents has a width of from about 200 nm to about 500 nm.

In particular embodiments, detecting the analyte comprises (i) exciting the one or more fluorescent sensors in the plurality of oblong optode sensing agents with an excitation energy emission from an energy emission device and (ii) detecting fluorescent energy emitted by the one or more fluorescent sensors in the plurality of oblong optode sensing agents.

In further embodiments, the energy emission device is a handheld device. In other embodiments, the semipermeable membrane comprises poly(2-hydroxyethyl methacrylate). In still more embodiments, the core has a diameter of less than or equal to about 100 nm. In other embodiments, the semipermeable membrane has a thickness of about 50 nm.

In some embodiments, the core further comprises a plasticizer.

In certain embodiments, implanting a plurality of oblong optode sensing agents comprises injecting the plurality of oblong optode sensing agents into the tissue. In particular embodiments, the tissue is selected from the group consisting of epidermal, muscular, ocular, endothelial

Other aspects disclosed herein relate to methods of detecting an analyte in a sample. Such detection is performed in vitro. As with detection in a tissue, each oblong sensing agent comprises: i) a core having one or more fluorescent sensors configured to bind to the analyte, and ii) a semipermeable membrane. In addition, the method of in vitro detection comprises contacting the plurality of oblong sensing agents with the analyte and detecting the analyte in the tissue.

In certain embodiments, the analyte is selected from the group consisting of electrolytes, salts, hormones, steroids, small molecules, drugs, and saccharides. In other embodiments, the core further comprises a polymer. In other embodiments, the semipermeable membrane comprises a hydrogel. In particular embodiments, the semipermeable membrane comprises a biocompatible hydrogel. In more particular embodiments, the semipermeable membrane is permeable to the analyte and impermeable to the one or more fluorescent sensors.

In certain embodiments, the core comprises one or more polymers selected from the group consisting of polyvinyl chloride, polylactic co-glycolic acid, methacrylate, and polycaprolactone

In other embodiments, the plurality of oblong optode sensing agents has a circular cross-section. In still other embodiments, the plurality of oblong optode sensing agents has a rectangular shape. In further embodiments, the plurality of oblong optode sensing agents has a length of about 40 μm to about 60 μm. In still further embodiments, the plurality of oblong optode sensing agents has a diameter of from about 200 nm to about 500 nm. In still more embodiments, the plurality of oblong optode sensing agents has a length of about 40 μm to about 60 μm. In yet more embodiments, the plurality of optode sensing agents has a width of from about 200 nm to about 500 nm.

In particular embodiments, detecting the analyte comprises (i) exciting the one or more fluorescent sensors in the plurality of oblong optode sensing agents with an excitation energy emission from an energy emission device and (ii) detecting fluorescent energy emitted by the one or more fluorescent sensors in the plurality of oblong optode sensing agents.

In further embodiments, the energy emission device is a handheld device. In other embodiments, the semipermeable membrane comprises poly(2-hydroxyethyl methacrylate). In still more embodiments, the core has a diameter of less than or equal to about 100 nm. In other embodiments, the semipermeable membrane has a thickness of about 50 nm.

In some embodiments, the core further comprises a plasticizer.

In certain embodiments, implanting a plurality of oblong optode sensing agents comprises injecting the plurality of oblong optode sensing agents into the tissue. In particular embodiments, the tissue is selected from the group consisting of epidermal, muscular, ocular, endothelial, mucosal, dermal, subcutaneous, and organ tissues.

### DESCRIPTION OF THE FIGURES

The following figures are presented for the purpose of illustration only, and are not intended to be limiting:

FIG. 1 shows a schematic of a cylindrical optode sensing agent.

FIGS. 2A and 2B show scanning electron micrographs of cylindrical optode sensing agents in a dehydrated state.

FIGS. 2C and 2D show transmission electron micrographs of the cylindrical optode sensing agents in a dehydrated state. Note the semipermeable membrane (light region) compared to the inner core (dark region).

FIG. 3A is a representation of a mold used to make the cylindrical optode sensing agents.

FIG. 3B is a representation that shows the coating of the mold with a hydrogel material. The coating is performed by initiated Chemical Vapor Deposition.

FIG. 3C is a representation that shows the filling of the pores of the mold with an optode material to form a core.

FIG. 3D is a representation that shows the removal of excess coating material and optode material.

FIG. 3E is a representation that shows the addition of the hydrogel material to cap the ends of the optode sensing agents.

FIGS. 4A is a confocal, fluorescent image of an optode sensing agent lacking a semipermeable membrane. The sensing agent is interacting with an analyte.

FIG. 4B is a confocal, brightfield image of the optode sensing agent of FIG. 4A.

FIG. 4C is an overlay of the images of FIG. 4A and FIG. 4B.

FIG. 5A is a confocal, brightfield image of the optode sensing agents having semipermeable membranes.

FIG. 5B is a confocal, fluorescent image of the optode sensing agents having semipermeable membranes.

FIG. 6 shows a plot of optode sensing agents (“microworms”) and nanoparticles (“spheres”). The surface-to-volume ratio is plotted against the effective hydrodynamic radius of the sensing agents and nanoparticles.

FIG. 7 is graph of the response of optode sensing agents (“microworms”) and nanosensors to sodium. Normalized fluorescent intensity α is plotted against the log of the sodium concentration for microworms (♦) and nanosensors (▪). Shown is the average with standard deviation for three measurements. Kd values are obtained by fitting the data to a sigmoidal function and values of 82 and 97 mM are obtained for nanosensors and microworms, respectively.

FIG. 8 is a graph of normalized fluorescent intensity of injected spots over time of optode sensing agents (“microworms”) (blue) and nanosensors (red). Shown is the average with standard deviation for three spots of each type of particle. The decrease in the nanosensor fluorescence intensity is due to the diffusion of the nanosensors away from the injection site. Over a time range of 1 h, no significant diffusion of the microworms is observed.

FIG. 9 shows an in vivo demonstration of optode sensing agents for sodium sensing. Optode sensing agents are subcutaneously located in the two injection spots on the left side of each mouse (I). Nanosensors are subcutaneously located in the two injection spots on the right side of each mouse (II). Each mouse is separated by a green bar to indicate that there were two different imaging conditions, I and II, for the two types of sensors to limit image saturation. The increased background in imaging condition I is due to the longer exposure times used to image the optode sensing agents. This method enabled imaging of both types of sensors in the presence of the autofluorescent noise.

FIG. 10 is a picture of a case that allows for the use of a portable electronic device as a fluorescence detection device.

FIG. 11 is an image of a hydrogel structure was filled with green fluorescent protein (GFP). The template was etched away and optode sensing agents minimally isolated to display GFP fluorescent core. Images were acquired with a Zeiss confocal microscope 488 nm laser line.

### DETAILED DESCRIPTION OF THE INVENTION

### 1. Oblong Optode Sensing Agents

The patent and scientific literature referred to herein establishes knowledge that is available to those of skill in the art. The issued US patents, allowed applications, published foreign applications, GenBank accession numbers, and references that are cited herein are hereby incorporated by reference to the same extent as if each was specifically and individually indicated to be incorporated by reference.

According to aspects of the present disclosure, an oblong optode sensing agent comprises a core and a semipermeable membrane. As used herein, the term “oblong” means having a length longer than a width or diameter. As used herein, the term “sensing agent” means a compound, nanoparticle, or substance that emits light when contacted by light or electromagnetic radiation. Such oblong optode sensing agents advantageously allows the sensors to remain at site of administration, while allowing the sensors to detect rapidly analytes. The optode sensing agent can have a circular cross-section. The optode sensing agent can have a rectangular shape. Exemplary shapes include rectangles, elongated cylinders having a diameter shorter than the length of the cylinder, oblong structures, parallelepiped structures, rhomboid structures, and elliptical structures. Generally, any structure that provides a high aspect ratio for the sensing agent is within the scope of the invention. By “high aspect ratio,” it is meant that the structures disclosed herein have lengths that are longer than their widths.

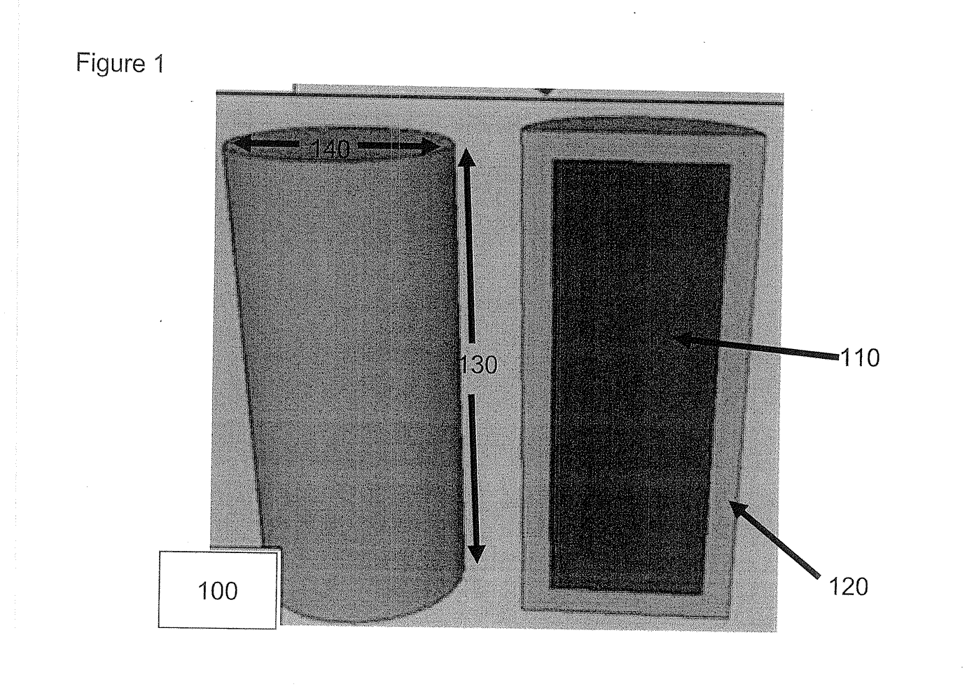

Referring to FIG. 1, a cylindrical optode sensing agent 100 is shown. In this embodiment, the optode sensing agent has a circular cross-section. The optode sensing agent 100 comprises a core 110 and a semipermeable membrane 120. The optode sensing agent 100 has a length 130 that is greater than the diameter 140 of the cylinder. For instance, the optode sensing agent 100 can be about 40 μm to about 60 μm. The optode sensing agent 100, on the other hand, has a diameter of between about 200 nm to about 500 nm. In still other embodiments, the optode sensing agent 100 has a diameter of about 500 nm to about 1000 nm. Notably, other optode sensing agents that are not cylinders can have the same dimensions. For example, rhomboid structures have widths rather than diameters. Therefore, the width of such structure can be about 200 nm to about 500 nm and about 500 nm to about 1000 nm.

The core 110 comprises sensors that are capable of binding to an analyte. In certain aspects, the core comprises one or more sensors configured to bind to an analyte. The sensors can bind electrolytes, salts, hormones, steroids, small molecules, drugs, and saccharides. The sensors disclosed herein can bind to sodium, potassium, bicarbonate, or other electrolytes. The sensors can also bind to magnesium, calcium, or other salts. The sensors can be to transition metals such as iron, manganese, nickel, and cobalt. The sensors can also bind to hormones such as testosterone, estrogen, or other hormones. The sensors can also bind to cholesterol and other cholesteryl-based structures. The sensors can further bind to small molecules having molecular weight of less than 1 kD. In certain embodiments, the small molecules can have a molecular weight of less than 2 kD.

In addition, the sensors disclosed herein can be fluorescent sensors. In such embodiments, the fluorescent sensors can be excited by any wavelength of light between 400 nm to 800 nm. In certain embodiments, the excitation wavelength of light is between 450 nm to 700 nm.

In particular embodiments, the core 110 comprises a polymer. The polymer can be polyvinyl chloride, polylactic co-glycolic acid, methacrylate, and polycaprolactone. The core 110 can further have a plasticizer. The plasticizers are additives that increase the plasticity or fluidity of the core 110. Any biocompatible plasticizer that does not affect the one or more sensors ability to detect an analyte can be used. In certain embodiments, the core 120 comprises about 33% plasticizer. In other embodiments, the plasticizer is less than 33% of the total material. In certain embodiments, the semipermeable membrane comprises a hydrogel.

Returning to FIG. 1, the semipermeable membrane 120 comprises a biocompatible hydrogel. The semipermeable membrane is permeable to analytes, but it is impermeable to the sensors and optode polymer. Exemplary biocompatible hydrogels for use as a semipermeable membrane include poly(2-hydroxyethyl methacrylate). The semipermeable membrane can be a confused membrane. As used herein, “confused membrane” means a membrane that is made of both hydrophobic and hydrophilic polymers that are mixed to create random hydrophobic and hydrophilic regions. Hydrophilic polymers include acrylics include acrylic acid, acrylamide, and maleic anhydride polymers and copolymers, amine-functional polymers include allylamine, ethyleneimine, oxazoline, and other polymers containing amine groups in their main- or side-chains. In addition, hydrophilic polymers include vinyl alcohols, polystyrenesulfonate, ethers, and vinyl acids. Such polymers can be obtained commercially from, for example, Sigma-Aldrich Corp. (St. Louis, Mo.). Exemplary hydrophobic polymers include lipophilic substances, hydrophobic modified polymers, and aliphatic polymers.

In certain embodiments, the optode sensing agent has a core that has a diameter of less than or equal to about 100 nm. In additional embodiments, the optode sensing agent has a semipermeable agent that has a thickness of about 50 nm.

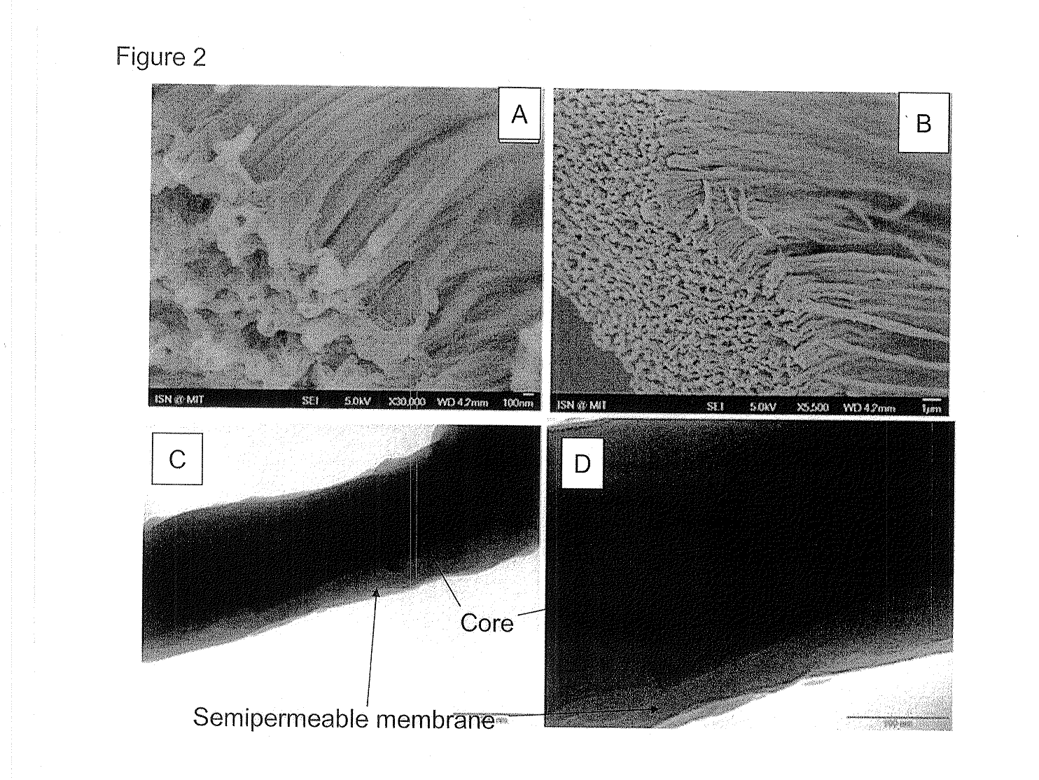

FIGS. 2A-D shows transmission and scanning electron micrographs of optode sensing agents in their dehydrated state. The micrograph images of single optode sensing agents in the dehydrated state confirm that the core is filled with optode.

### 2. Methods of Making Oblong Optode Sensing Agents

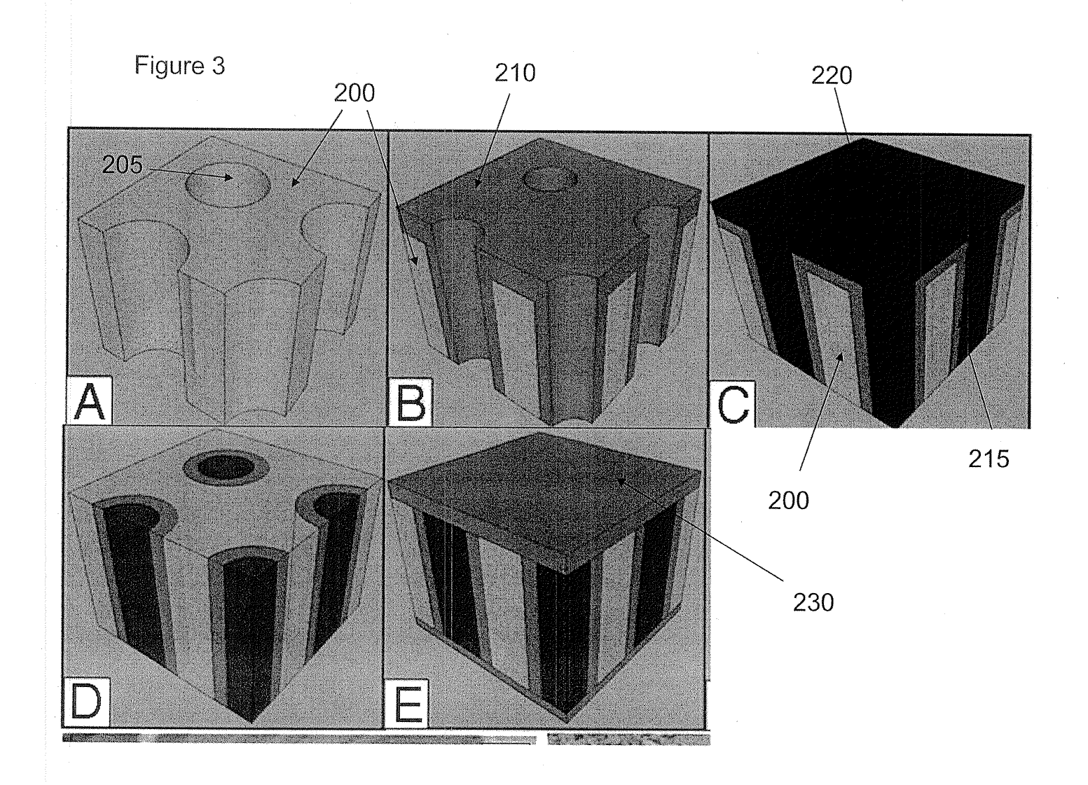

Aspects disclosed herein include methods of making oblong optode sensing agents. In certain embodiments, the methods comprise providing a mold 200 comprising one or more pores (FIG. 3A). The mold 200 can be a porous mold comprising multiple nanopores 205 having diameters of less than or equal to about 500 nm. The mold 200 can also comprise pores 205 having diameters of less than or equal to about 1000 nm. The mold 200 can be composed of any material that can be etched away from the made optode sensing agents. In other embodiments, the optode sensing agents can be sonicated from the mold. In particular embodiments, the mold 200 can be composed of aluminum oxide such as anopore aluminum oxide.

The method of making oblong optode sensing agents further comprises coating the interior surfaces of the one or more pores 205 with a first material, the first material forming a semipermeable membrane. Referring to FIG. 3B, the mold 200 is provided the first material through deposition of a first material to form a coating 210. The first material can be a hydrogel material mixed with one or more volatile substances (e.g., THF) to help with the deposition of the hydrogel material. Various deposition techniques can be used. For instance, the coating can be provided to the mold surface through chemical vapor deposition (“CVD”) (see, e.g., Jaeger, Richard C. (2002). “Film Deposition.” Introduction to Microelectronic Fabrication. Upper Saddle River: Prentice Hall.). In a CVD process, one or more volatile precursors are placed on a surface. The precursors react and/or decompose on the mold surface to produce the desired deposit. Frequently, volatile substances are generated. Such substances are removed by gas flow through the reaction chamber where the deposition occurs.

After the coating 210 is applied, a second material 220 is applied to the one or more coated pores 215 such that the second material fills the one or more coated pores 215, the second material comprising one or more sensors (FIG. 3C). The second material 220 is the optode solution, which is filled in the coated pores 215 of the mold 200. This forms the core of the optode sensing agent. The excess second material 220 and the first material comprising the coating 210 is etched away (FIG. 3D). A final hydrogel layer 230 is deposited on both sides of the template to cap the optode sensing agent (FIG. 3E). The optode sensing agents are allowed to form using techniques known in the art.

As the final step, the mold 200 is dipped in an acid solution (e.g., HCl) to etch the mold 200 away from the optode sensing agents (FIG. 3F).

In particular embodiments, the methods involve hydrogel deposition that is performed in an anopore aluminum oxide (“AAO”) mold. The conformal deposition inside the pores of the AAO mold is achieved by tuning the deposition parameters to keep the ratio of the monomer partial pressure (Pₘ) to monomer saturation pressure (Pₛₐₜ) low (Ozaydin-Ince G, Gleason K K (2010) Chem Vap Deposition 16:100-105; Baxamusa S H, Gleason K K (2008) Chem Vap Deposition 14:313-31). Although temperature of the AAO mold is difficult to control, by maintaining low reactor pressures, low Pₘ/Pₛₐₜ values of 0.1-0.3 can be achieved which improves the conformality. Transmission electron microscopy (TEM) images of single optode sensing agents in the dehydrated state confirm that the optode fills the hydrogel tubes (FIGS. 2A-B). The darker core is the optode, whereas the lighter outer shell is the hydrogel coating.

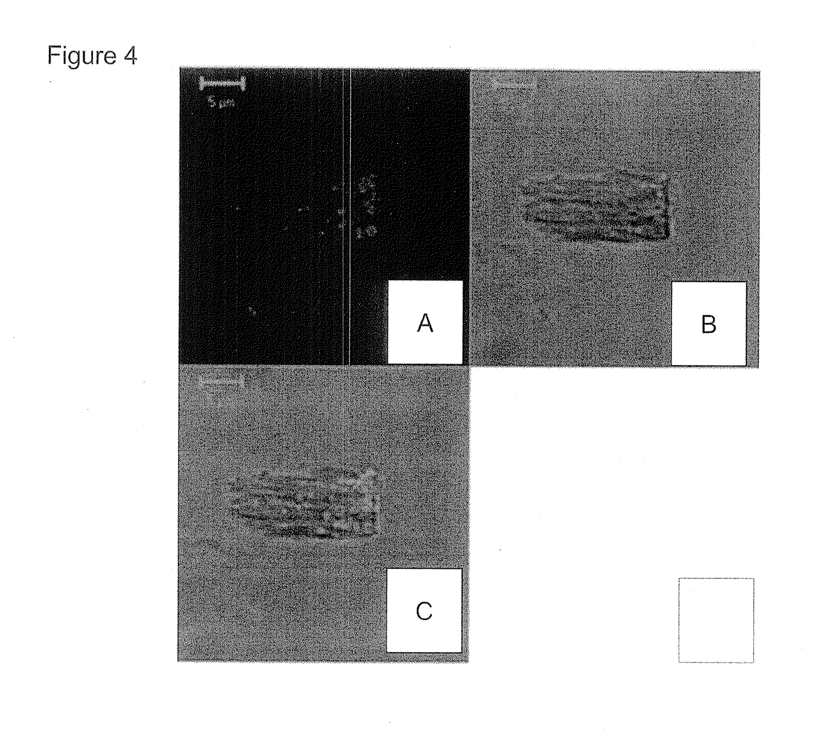

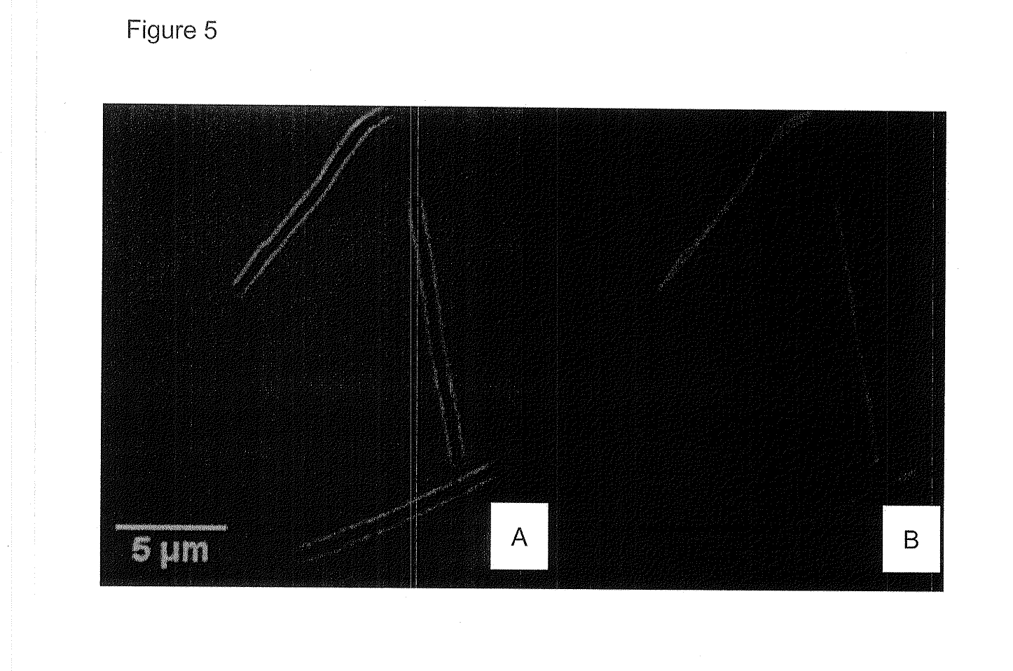

FIGS. 4A-C show examples of optode sensing agents made with the methods described herein. The optode sensing agent shown in FIGS. 4A-4C lacks the semipermeable membrane. As shown in FIGS. 4A-C, the optode sensing agents assume circular shapes when made in molds having circular pores. FIGS. 5A and 5B shows optode sensing agents that are completed. The strong fluorescent image seen in FIG. 5B confirms that the outer hydrogel layer does not affect the fluorescence of the optode sensing agents.

### 3. Detecting Oblong Optode Sensors

Aspects disclosed herein include methods of detecting an analyte in a tissue of a subject. The method comprises implanting a plurality of oblong optode sensing agents in the tissue. The implanting can involve surgical implantation, injection into tissue, or administering to the surface of a tissue. Furthermore, the methods comprise detecting an analyte in the tissue. Exemplary tissues include epithelial tissues such as skin, endothelial tissues such as blood vessel walls, organ tissues, ocular tissues, muscle tissues, and mucosal tissues.

In embodiments in which the optode sensing agents are administered to a subject. The optode sensing agents can be administered in solutions that allow for administration of the optode sensing agents to a site of interest. The only limitation on such solutions is that they must not interfere with the functioning of the optode sensing agents.

In certain embodiments, the optode sensing agents are tattooed onto the subject. Such tattooing has been disclosed previously in U.S. Appl. Pub. No. 2009/0155183 A1, incorporated by reference herein. In such embodiments, the intra- and extra-cellular optode sensing agents reside under the tissue. In certain embodiments, popular, commercially available portable electronic devices with optical readers can be transformed into a diagnostic instrument that can also communicate with care providers and provide assistance in emergency cases.

In additional embodiments, the optode sensing agents are biocompatible to prevent an immune response. The response can be specific to the concentration of the molecule or ion being measured. The response can be quantitatively measurable by the portable electronic imaging device.

Other aspects include methods of detecting an analytes comprising providing a plurality of oblong sensing agents into a sample. In these aspects, the oblong optode sensors are provided to a sample isolated from a patient. For instance, the samples are isolated by biopsy or other minimally invasive means. The samples can also be isolated by surgical procedures. The isolated tissues can be processed using techniques known in the art to obtain a sample amenable to ELISA procedures or other fluorescent-based detection procedures.

Further embodiments entail providing or attaching optode sensing agents into a well, such as on a multiwell plate, tubing, chip, membrane, or surface that contacts samples in which analytes are present. In these embodiments, the optode sensing agents interact or bind to analytes. Subsequently, the optode sensing agents that have interacted or bound to analytes will fluoresce when contacted with an excitation light. Thus, the sensing agents can detect one or more analytes that have passed across a surface. Methods of attaching polymers to solid supports such as microchips are known in the art, e.g., Hynd M, et al. Functionalized hydrogel surfaces for the patterning of multiple biomolecules. Biomaterials. (2007) 81:347-54, the disclosure of which is incorporated by reference.

Optode sensing agents can be continuous monitoring agents in the disclosed methods. To be a continuous monitoring agent, the response time of the optode sensing agents should be fast. The response time of the sensor is completely dependent on diffusion within the optode and decreases below seconds as the size decreases to microns (Bakker E, Bühlmann P, Pretsch E (1997) Chem Rev 97:3083-3132). Theoretical response times of the nanosensors and the optode sensing agents can be estimated by solving the diffusion equation for a sphere (see, e.g., Crank J (2004) The Mathematics of Diffusion (Oxford Univ Press, New York), pp 69-104) and a rod (see, e.g., Crank J (2004) The Mathematics of Diffusion (Oxford Univ Press, New York), pp 69-104).

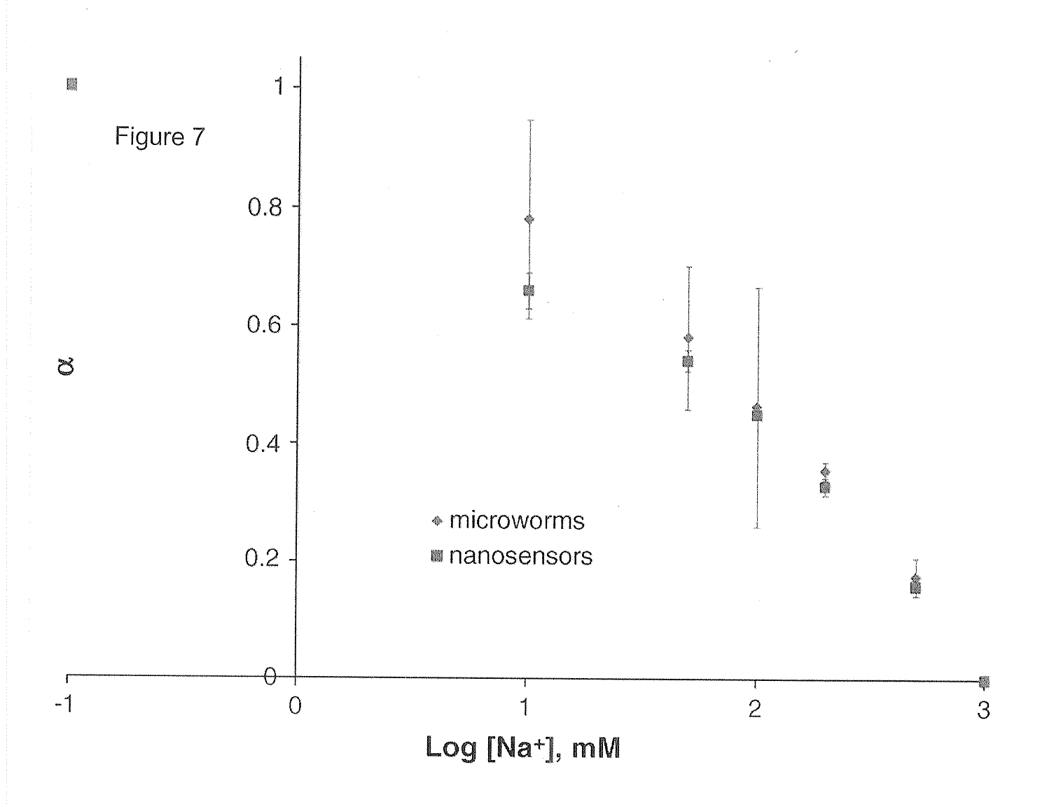

The response mechanism of the sensors to sodium has been previously explained (Bakker E, Bühlmann P, Pretsch E (1997) Chem Rev 97:3083-3132). Briefly, the optode consists of a plasticized polymer that creates a hydrophobic environment in which a pH sensitive fluorophore, an ionophore specific for sodium, and a charge neutrality molecule are contained. Selective uptake into the optode by the ionophore brings a positive charge into the polymer resulting in a loss of a hydrogen ion to balance the charge. This hydrogen ion loss changes the protonation state of the fluorophores and thus the optical properties of the optode. The optical response of nanosensors and optode sensing agents to sodium was determined with a plate reader. The intensity of a given sodium concentration was normalized according to (α=Imax−I [Na+])(Imax−Imin), where Imax is the intensity of the nanosensors or the optode sensing agents at zero sodium, Imin is the intensity at 500 mM sodium, and I [Na+] is the intensity at the concentration of interest. The response data, α, was plotted against the log of the sodium concentration, in millimolar, with the log of zero sodium set to −1 for curve fit analysis. The response data for both the optode sensing agents and the nanosensors are shown in FIG. 7. Sigmoidal curves are fit (Origin) to the data and using the software the Kd is calculated as 82 mM for the nanosensors and 97 mM for the optode sensing agents.

This Kd is satisfactory within resting interstitial sodium concentration, which is roughly 130 mM. Considering the error bars in response (FIG. 7), the difference in the Kd values is insignificant, indicating that the response of the optode sensing agents is the same as the nanosensors.

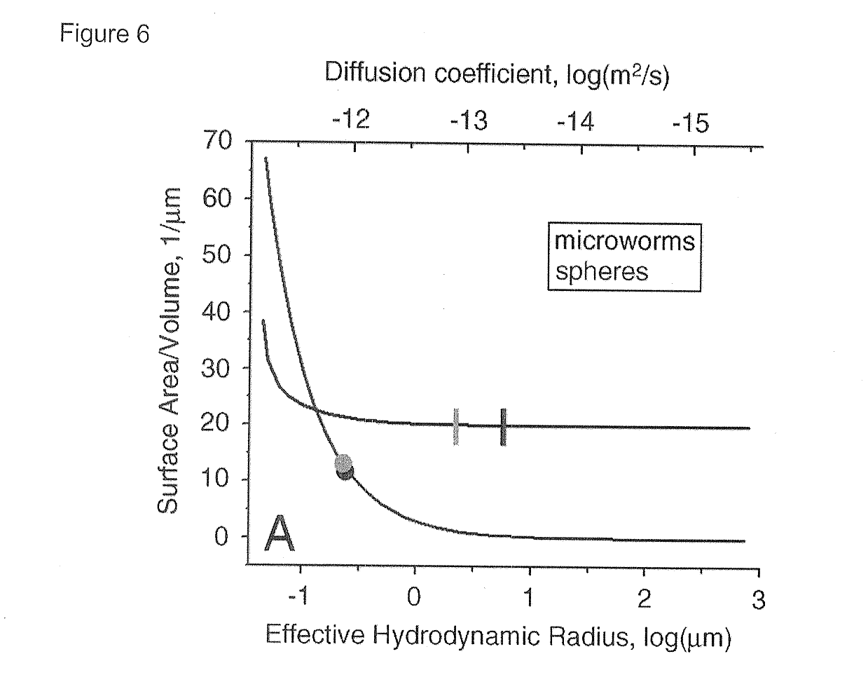

Additionally, optode sensing agents can be in vivo monitoring agents. For continuous in vivo monitoring, the diffusion of the sensors should be minimized. The diffusion coefficient, D, can be calculated using the Stokes-Einstein equation D=kT/6πηRₕ, where k is Boltzmann's constant and η is the viscosity of water. The hydration radius (Rₕ) of the optode sensing agents can be found from the radius of gyration (R_{g}) (30), here R_{g}=1.732 Rₕ. The R_{g} of a cylinder depends on the length and radius [R_{g}=L²/12+r²/2^{1/2}], where L is the length and r is the radius. For aspect ratios L/r>>1 the radius of the optode sensing agent contributes little to R_{g} and can be ignored in this case because L/r=400. For a length of 40 μm and a radius of 100 nm, the Rₕ of the optode sensing agent is 6.67 μm. The surface-area-to-volume ratio of the optode sensing agent is 20 μm⁻¹, whereas the surface-area-to-volume ratio of a sphere with an Rh of 6.67 μm is 0.45 μm¹, shown in FIG. 6. At smaller effective Rₕ, the surface-area-to-volume ratio is actually greater for spheres. However, once the effective Rₕ is above 150 nm, the optode sensing agent structure provides an improved surface-to-volume ratio.

For optode sensing agents of length 40 μm, the theoretical diffusion coefficient 30° C. was 4.1×10⁻¹⁴ m²/s. The theoretical diffusion coefficient 30° C. of the spherical nanosensors with a diameter of 272 nm was 2.0×10⁻¹² m²/s. These are shown in FIG. 6 as a blue line and a blue circle. By matching the diameter of the nanosphere to that of the optode sensing agent, the response times to sodium will be similar for both shapes. For the given geometrical structure and dimensions, the diffusion coefficient of the optode sensing agents is calculated to be approximately 50 times smaller than that of the nanosensors. The experimental values of the diffusion coefficients were measured by dynamic light scattering (DLS) experiments and are shown as a green line and green circle in FIG. 6. FIG. 6 shows the surface-area-to-volume ratio of optode sensing agents (black) and nanosensors (red), plotted as a function of the diffusion constant, and thus hydrodynamic radius (FIG. 6). Shown are the theoretical values for the sizes used here for optode sensing agents (blue line) and nanosensors (blue dot), as well as the experimentally measured diffusion coefficient values, green line and green dot for optode sensing agents and nanosensors, respectively. The average diffusion coefficient of the optode sensing agents at 30° C. was 1.54+/−0.33×10⁻¹³ m²/s. The spherical nanosensors of 271.6+/−12.4-nm diameter, on the other hand, had an average diffusion coefficient of 1.699+/−0.237×10⁻¹² m²/s at 30° C.—an order of magnitude larger than that of the optode sensing agents. The difference between the theoretical calculation and the DLS experiment results for the diffusion coefficient of the optode sensing agents may be related to the variations in the nanoworm sizes (polydispersity of 0.74+/−0.2) or the bending or twisting of the optode sensing agents during diffusion which changes the hydrodynamic radius.

### 4. Detection Devices

As discussed above, the methods of detection can be utilized to cause excitation of the fluorescent molecules. In certain embodiments, the fluorescence emitted can be captured using devices useful in fluorescence imaging. For instance, the images of the fluorescence can be captured on a camera on a portable electronic device. Furthermore, the optode sensing agents can be detected using standard ELISA techniques in vitro. In other embodiments, the optode sensing agents are detected using devices known in the art. For instance, the IVIS Spectrum Imaging System (Caliper Lifesciences, Hopkinton, Mass.) is useful for in vivo detection of fluorescence.

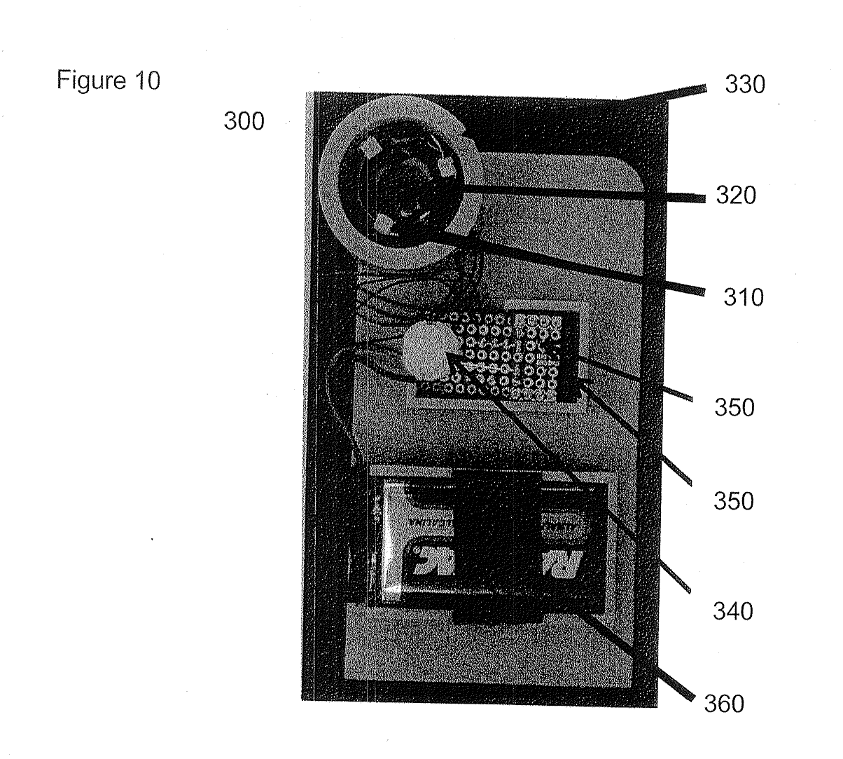

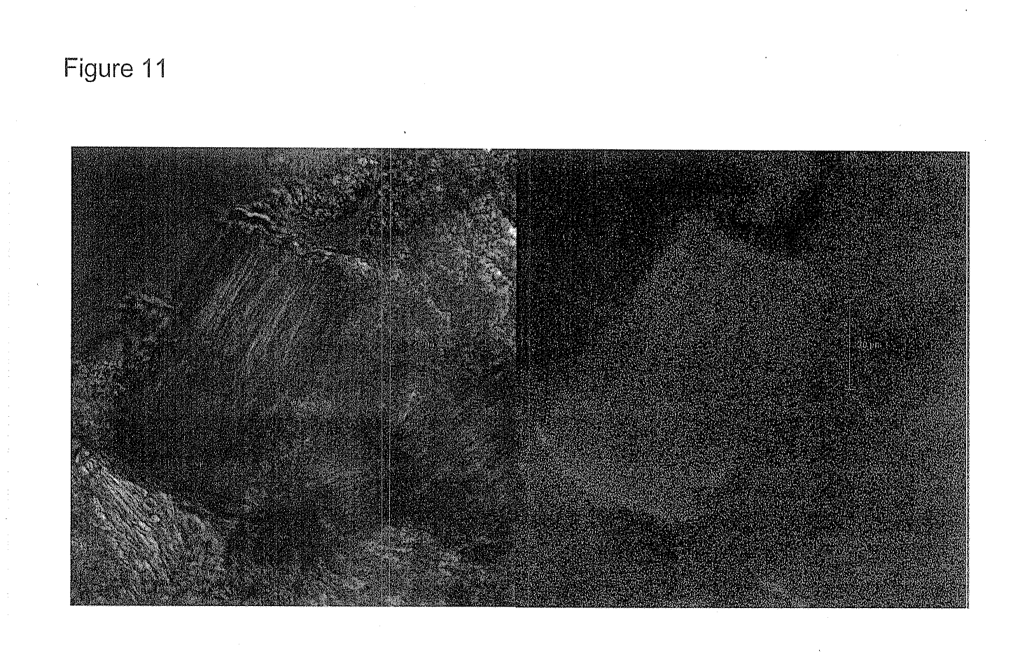

In certain embodiments, the optode sensing agents are detected using a portable electronic device converted into a handheld diagnostic device. An example of a portable electronic device converted to a handheld diagnostic agent is shown in FIG. 10. The electronic device would be attached to a case 300. The case 300 is capable of emitting an excitation wavelength of light through light emitting diodes (“LEDs”) 310 positioned upon the outer surface of the case 300. As shown in FIG. 10, the LEDs 310 are disposed on the case 300 so as to allow the user to direct light emitted from the LEDs 310 onto a sample (e.g., biological, chemical, or environmental) or in a tissue. The excitation wavelength emitted by the LEDs 310 contacts the optode sensing agents in a sample or in a tissue.

The case 300 also has an optical filter 320 positioned on it. In certain embodiments, the optical filter 320 is a fluorescent filter that is designed to filter out the excitation wavelength emitted by the LEDs 310, thereby allowing the fluorescent light emitted from the fluorescent sensors to contact a lens of a camera on the portable electronic device (not shown) without excitation light confounding the image. Fluorescent filters are known in the art and can be obtained commercially from suppliers Chroma Technology Corp (Bellows Falls, Vt.).

In certain embodiments, the optical filter 320 is positioned on a chamber 340 attached to the case 300. The chamber 330 is attached to the case 300 in such as manner to allow for the optical filter 320 to be positioned over the lens of the camera on the portable electronic device. In this way, the optical filter 320 allows fluorescent light from the sensors to illuminate the lens of the camera. In particular embodiments, the chamber 330 is a box structure comprising an optical filter defining one surface of the chamber directly in front of the lens of the camera of the portable electronic device. Such chambers have four sides that do not allow light to be transmitted to the lens of the camera. Thus, the only light that contacts the lens of the camera is the fluorescent light emitted from the fluorescent sensors.

The case can be made of known materials and using known methodologies. The chamber can be made of the same materials used to make the case. It can be produced separately and attached to the case after the case has been produced. For instance, the case and/or chamber can be made of plastics such as polyethylene, polypropylene, polystyrene, and polyvinyl chloride. The case and/or chamber can also be made of metals such as aluminum, and metal alloys. The case and/or chamber can also be made of fiber glass.

The case can be constructed through rapid prototyping. For example, injection molding techniques can be used or any available techniques for building cases for portable electronic devices (see, e.g., Bryce. Plastic Injection Molding: Manufacturing Process Fundamentals. SME, 1996. pg. 43-44).

In additional embodiments, the case 300 comprises an optional push button 340 to activate and deactivate the LEDs 310. In some embodiments, the case 300 comprises a microcontroller 350 that controls the LEDs 310. The microcontroller can be an Arduino microcontroller 350. The microprocessor contains code to allow the case 300 to control the LEDs 310. The case can also comprise a power source, such as a battery 360.

Alternative portable electronic devices could use a camera in the case 300 that can transmit the images to the mobile phone for analysis. Also, the device can be powered by a smaller battery with a voltage gain or by the portable electronic device itself. Data and power transmission can alternatively occur though wire connecting the microcontroller to the portable electronic device.

In particular embodiments, a kit is provided with the optode sensing agents and reference agents for calibrating detection devices. The kits can comprise packaging that is fluorescent and can be used to calibrate the detection devices used to detect the fluorescent sensors. For instance, the kit houses two compartments of optode sensing agents at known analyte concentrations. When a fluorescent image of the packaging is taken, the fluorescence intensity can be measured. Because the analyte concentration is known, and the calibration curve of this specific lot of sensors has been determined based on a QR code, the intensity measurement can be used to adjust the calibration curve to the particular device. In some embodiments, two measurements at known concentrations of analyte—such as sodium—can allow a software application to set the calibrated response to the correct fluorescence ration. Such software calibration is known in the art.

This approach will completely calibrate the sensors at each measurement point to eliminate drift in sensor functionality or other measurement artifacts. The intensities at each measurement will be recorded by the software application and alert patient.

The fluorescent image of the sensors in the packaging can be used to measure the fluorescence intensity of the sensing agents; this calibrates the tissue of the subject. By taking an image of these sensors, both before and after injection, a software program can determine how the tissue alters the fluorescence. This known alteration of the fluorescence can then be used to determine the correct analyte concentration from the fluorescence of the analyte sensors. This can be performed using the following equations:

$$Z = \frac{{R}_{Packaging}^{Reference}}{{R}_{Injected}^{Reference}} , {R}_{True}^{Na} = Z * {R}_{Measured}^{Na}$$

“Z” is a correction factor that is calculated by dividing the intensity “R” of the reference sensors in packaging by the value immediately after the sensors have been injected. The packaging value will be stored for the lifetime of the sensors and used to create a new “Z” at each reading. The true “R” of the sodium sensors will then be determined by multiplying “Z” by the measured “R” of the sodium sensors.

In medical diagnostic embodiments, a doctor or a patient is provided with a kit comprising the fluorescent sensors and a light-protective cover. After a light-protective cover has been removed the patient can take two images, one bright field and one fluorescent. The bright field image will allow the disclosed system to register the sensors by recognizing the QR code. A QR code is similar to a barcode and is an increasingly common way to relay information. The code will provide the lot number of the optode sensing agents which can be used to determine expiration date and calibrated response of the sensing agents. The optode sensing agent lot can be calibrated in the factory and this information can be transferred to the portable electronic device.

In further embodiments, reference sensors are injected into the tissue that will be imaged each time the analyte concentration is determined. Therefore, at each measurement the correction factor obtained from the fluorescence of these sensors will be adjusted. This can remove error that may occur from sensing agent degradation, photobleaching, tissue autofluorescence or other biological or physical artifacts when sodium concentration is measured.

After the doctor or patient has taken images of the packaging, the sensors will be injected into the tissue or surgically implanted into the tissue directly. One of the compartments containing optode sensing agents is administered to the tissue. In certain embodiments, reference sensors are administered to another position in the tissue. There are several minimally-invasive injection technologies that are FDA-approved and available on the market. These injection technologies are mainly designed for drug delivery devices or vaccine administration, but may be adapted for sensor delivery into the intradermal space. For example, in March 2011 the FDA cleared the first intradermal needleless injection system by PharmaJet® for vaccine delivery. These injection systems can delivery up to 100 μl of solution to the intradermal space using a high pressure system. Microneedle delivery systems can also be used to deliver sensors into the intradermal space without triggering the nerve fibers. After the two spots have been injected, a simple mark from a stamp in the packaging will indicate where the injections have been made as well as the orientation of the two spots.

In certain embodiments, a patient monitors analyte concentrations. Any time the patient wants to measure his or her analyte levels, the patient takes an image of the injection spot. This can provide an accurate measurement in real time that is pain free. After the sensors have been injected they can last for one week before they need to be replaced. At some point after a week, the sensors will begin to biodegrade and be removed naturally by the body. Removal can occur by renal elimination and sloughing off of sensor material when the dermis layer of the skin is replaced. To replace the sensors the patient simply uses a new package of sensors, takes the initial images, and injects the sensors in a different spot in their skin. There will be no permanent effects from the sensors so the same spot could eventually be used again.

In an illustrative embodiment, the fluorescent sensors have multiple fluorescent excitation and emission wavelengths that can be used to determine fluorescent intensity. To achieve quantitative and accurate measurements, however, a ratio of two wavelengths must be used. This eliminates the dependency of the calculated sodium concentration on the number of sensors present, accounts for skin inhomogeneity, sensor injection depth, and possible photobleaching. Two emission wavelengths can be created by exciting the chromoionophore, for example, at 476 nm and collecting emission, for example, at the 570 nm and 670 nm peaks.

### EXAMPLES

### Materials

The monomer 2-hydroxyethyl methacrylate (HEMA, 99%, Aldrich), the cross-linker ethylene glycol diacrylate (EGDA, 98%, PolySciences), and the initiator tert-butyl peroxide (TBPO, 98%, Aldrich) were used as received. All chemicals used to prepare the optode were purchased from Sigma. The AAO membranes were purchased from Whatman, Inc. The pores of the membranes are 60 μm in length and 200 nm in diameter.

Sodium selective optode was created from the following compounds: 30 mg high molecular weight poly(vinyl chloride); 60 mg bis-2(ethylhexyl)-sebacate; 3 mg sodium ionophore X; 0.5 mg chromoionophore III; and 0.4 mg of sodium tetrakis[3,5-bis(trifluoromethyl)phenyl]borate. These components were brought up in 500 μL of THF.

### Initiated CVD System

Initiated CVD depositions were performed in a custom-built high-vacuum reactor with a base pressure of 10⁻⁴ torr. The monomer vapors were delivered to the reactor using needle valves, whereas mass flow controllers were used to deliver nitrogen gas (N₂) and the initiator. The initiator molecules were thermally decomposed by a heated filament array that consists of 14 parallel ChromAlloy filaments (Goodfellow) that were placed 2 cm above the sample. The sample temperature was controlled by backstage cooling using a chiller and heater (Neslab, Thermo Scientific).

### Polymer Coating Deposition

The biocompatible hydrogel film, cross-linked p(HEMA), was deposited using iCVD. The flow rates of HEMA, EGDA, TBPO, and N2 used were 0.6, 0.15, 1, and 1 sccm, respectively. During depositions, the reactor pressure was kept at 200 mtorr. The filament and the substrate temperatures were maintained at 215 and 30° C., respectively.

### Nanosensor Fabrication

Nanosensors were prepared as previously described (Dubach J M, Harjes D I, Clark H A (2007) Nano Lett 7:1827-1831). Briefly, optode was diluted 50% with dichloromethane and added dropwise to 4 mL of aqueous solution, either PBS or 10 mM Hepes, containing 1,2-diacyl-sn-glycero-3-phosphoethanolamine-N-[methoxy(polyethyleneglycol)-550] (Avanti Polar Lipids). The solution was then mixed with a probe tip sonicator to form nanosensors (Branson).

### Fabrication of Optode Sensing Agents

For the fabrication of the optode sensing agents, AAO templates were first conformally coated with p(HEMAco-EGDA) hydrogel with a wall thickness of approximately 50+/−10 nm. The pores of the coated AAO templates were then filled with the optode solution. Optode was first diluted in 1 mL of THF in a glass dish inside a chamber with a THF bath to prevent rapid evaporation of THF in the optode solution. The coated AAO templates were placed facing down in the glass dish and allowed to float on the optode for 1-2 min. The AAO templates were then washed in a THF bath to rinse excess optode, inverted, and allowed to dry while resting on two glass micropipettes. Both sides of the templates were then iCVD coated with hydrogel to cap the ends of the tubes. As the final stage, the templates were etched in HCl and the optode sensing agents were released. To prepare the optode sensing agents for tests, they were suspended in 2 mL of water. This solution was sonicated using a probe tip sonicator (Branson) for 1 min, four times. The solution was then transferred to microcentrifuge tubes and spun at 500 rpm for 5 min. The supernatant was removed and mixed 1:1 with PBS or HEPES buffer. This solution was then spun at 10,000 rpm for 30 min. The supernatant was then removed and discarded and the remaining pellet was brought up in PBS or Hepes buffer and resuspended.

For the preparation of the control samples, the pores of the AAO templates with no iCVD coating were filled with the optode solution the same way as described for the optode sensing agents. After the optode inside the pores was dried, the AAO templates were etched in HCl solution to release the cylindrical sensors without polymer coating.

### Chemical Analysis

The compositional analysis of the deposited hydrogel layer was performed by Nexus 870 FTIR (Thermo Nicolet) equipped with a deuterated triglycine sulfate and thermoelectricity cooled detector. The analysis was done on the polymer films deposited on silicon. The spectra were acquired at 4 cm⁻¹ resolution and the number of scans was kept at 128. The cross-link density, R, of the hydrogel layer was calculated using the relation (Chan K, Gleason K K (2005) Langmuir 21:8930-8939)

R=[EGDA]/[HEMA]=[(A_{C═O}−rA_{O—H})/2]/rA_{O—H}

where A_{C═O} and A_{O—H} are the FTIR peak areas of the C═O stretching and O—H regions, respectively, for the copolymer films. The ratio r is of peak areas AC1/4O to AO—H for the linear p(HEMA) film. For the deposition parameters used in this study, the crosslinking density was calculated to be 0.19.

### Imaging

The fabricated optode sensing agents were imaged with a field emission gun SEM (JEOL J5M-6700F) instrument. The TEM analysis was performed with an FEI Tecnai F20. Confocal images were recorded on a Zeiss 510 meta confocal microscope. Optode sensing agents were imaged using a 633-nm He—Ne laser with emission at 680+/−10 nm using a 63×1.4 N.A. oil immersion objective.

### Size Analysis

Size measurements of nanoparticles and optode sensing agents were performed using dynamic light scattering (Zetasizer, Nano Series ZS90, Malvern Instruments). Measurements were made in triplicate at temperatures ranging from 15 to 35° C. at 5° increments.

### Sensor Response

Calibration was performed by diluting the 100 μL of optode sensing agents or nanosensors in 100 μL of 10 mM Hepes solution pH 7.4 with sodium chloride concentrations ranging from 0 to 1 M. The solutions were loaded into an optical bottom 96-well plate (VWR) in triplicate per sodium concentration. The fluorescence was read in a plate reader exciting at 633 nm and collecting emission at 680 nm (Spectramax Gemini EM, Molecular Devices).

### In Vivo Measurements

Optode sensing agent and nanosensor diffusion measurements were performed in nude, immune-compromised CD-1 mice (Charles River Labs). All animal procedures are approved by the Institutional Animal Care and Use Committee of Tufts University Medical School, Boston, Mass. Mice were anesthetized using Isofluorane. Twenty microliters of optode sensing agents or nanosensors in PBS solution were injected subcutaneously in the back of each mouse in multiple spots. The mice were then imaged using an IVIS 200 (Caliper Life Sciences). Brightfield and fluorescent images were taken at 5 min intervals for 60 min. Fluorescent images were acquired using the Cy5 filter settings. Two imaging conditions were used for fluorescent images, one for the optode sensing agents and one for the nanosensors. These images were taking back to back and therefore have the same time points. The conditions were changed between the two sensors to ensure that each sensor was producing similar fluorescent intensities compared to the other without saturating the image. Both types of sensors were exposed to both imaging conditions to prevent the effects of photobleaching causing differences in fluorescence efficiency. Regions of interest were analyzed using total efficiency and were background subtracted.

### Results

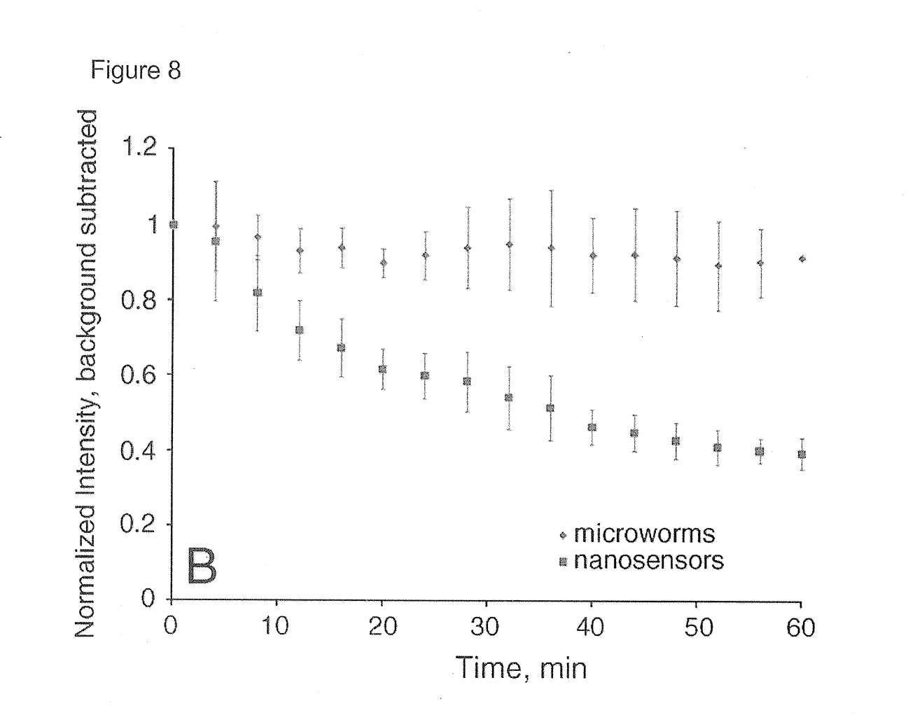

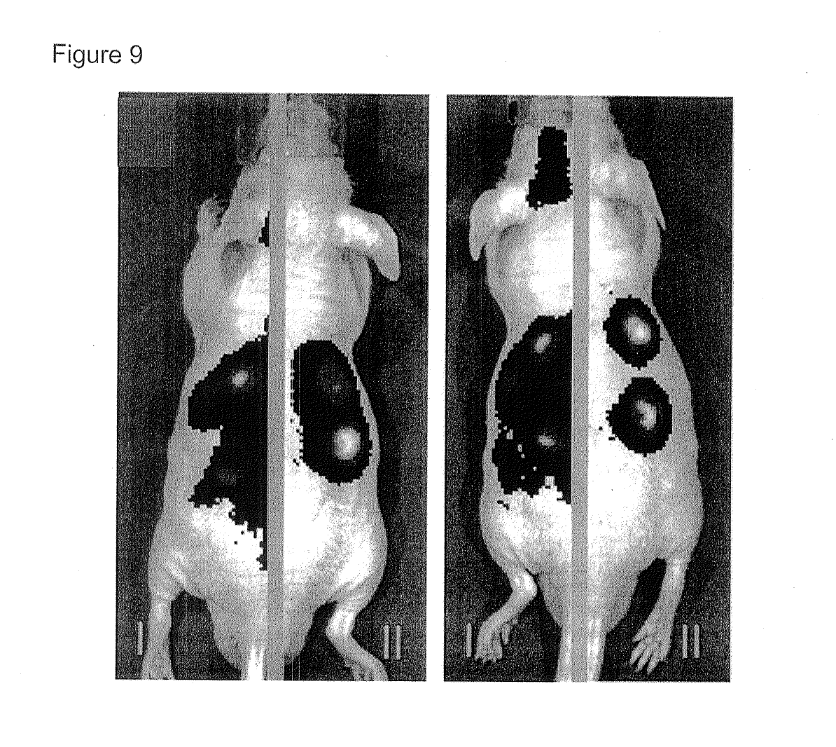

The theoretical advantages of the optode sensing agents were confirmed experimentally by subcutaneously injecting optode sensing agents and nanosensors in nude mice. The fluorescence efficiency of the sensors were tracked over time (FIG. 8). The fluorescence efficiency represents the total amount of fluorophore present and thus indicates that amount of sensor, either optode sensing agents or nanosensors, that is present in the region analyzed. This measurement therefore can be used to determine diffusion away from the spot of injection. The optode sensing agents show little diffusion away from the injection spot over the course of an hour, however, the nanosensors have significant diffusion as the efficiency decreases by roughly 60% (FIG. 9). Experiments were stopped at 1 h as the decrease in fluorescence efficiency of the nanosensors leveled and therefore an accurate comparison of retention in the injection spot could be made. Each sensor is made of the same components and has similar responses with respect to sodium and photobleaching. Also, nude, immune-compromised mice were used to eliminate the possible effects of an immune reaction to the sensors. Previous experiments have shown that similar nanoparticles do not elicit a noticeable immune response of the same time period (Billingsley K, et al. (2010) Anal Chem 82:3707-3713) and phagocytosis of particles with similar size and shape is not possible (Geng Y, et al. (2007) Nat Nanotechnol 2:249-255). Therefore, the difference in efficiency changes between the optode sensing agents and the nanosensors is solely due to diffusion of the sensors out of the injection spot. Sensor degradation via leaching was not considered because it has previously been shown that PEG-coated nanosensors will not leach components and are stable in solution for weeks (Dubach J M, Harjes D I, Clark H A (2007) Nano Lett 7:1827-1831). The diffusion properties of the optode sensing agents are better suited for measurement conditions over the course of 1 h. At this smaller diffusion rate, the optode sensing agent geometry has a much larger surface-area-to-volume ratio that allows for rapid responses to changes in the sodium concentration. Therefore, the optode sensing agents developed here have a more ideal geometry than spheres for use of in vivo monitoring.

Incorporating the sodium optode into the optode sensing agent sensor prevented diffusion of the sensor away from the spot of subcutaneous injection. Furthermore, the optode sensing agent shape and coating did not prevent optode interaction with the surrounding environment. Optode sensing agents present a biocompatible mechanism to immobilize polymer sensing material in vivo for continuous monitoring. This technology can be extended to immobilize any polymer with the capacity of adjusting the hydrogel coverage or cross-linking density to alter medium interaction.

We claim:

1. An oblong optode sensing agent, the oblong optode sensing agent comprising a core and a semipermeable membrane, wherein the core comprises one or more sensors configured to bind to an analyte.

2. The optode sensing agent of claim 1, wherein the one or more sensors covalently bind to the analyte.

3. The optode sensing agent of claim 1, wherein the one or more sensors are fluorescent sensors.

4. The optode sensing agent of claim 2, wherein the analyte is selected from the group consisting of electrolytes, salts, hormones, steroids, small molecules, drugs, and saccharides.

5. The optode sensing agent of claim 1, wherein the core further comprises a polymer.

6. The optode sensing agent of claim 1, wherein the semipermeable membrane comprises a hydrogel.

7. The optode sensing agent of claim 5, wherein the semipermeable membrane comprises a biocompatible hydrogel.

8. The optode sensing agent of claim 1, wherein the semipermeable membrane is permeable to analyte and impermeable to the one or more sensors.

9. The optode sensing agent of claim 5, wherein the core comprises one or more polymers selected from the group consisting of polyvinyl chloride, polylactic co-glycolic acid, methacrylate, and polycaprolactone

10. The optode sensing agent of claim 1, wherein the optode sensing agent has a circular cross-section.

11. The optode sensing agent of claim 1, wherein the optode sensing agent has a rectangular shape.

12. The optode sensing agent of claim 10, wherein the optode sensing agent has a length of about 40 μm to about 60 μm.

13. The optode sensing agent of claim 12, wherein the optode sensing agent has a diameter of from about 200 nm to about 500 nm.

14. The optode sensing agent of claim 11, wherein the optode sensing agent has a length of about 40 μm to about 60 μm.

15. The optode sensing agent of claim 14, wherein the optode sensing agent has a width of from about 200 nm to about 500 nm.

16. The optode sensing agent of claim 1, wherein the semipermeable membrane comprises a confused surface.

17. The optode sensing agent of claim 7, wherein the semipermeable membrane comprises poly(2-hydroxyethyl methacrylate).

18. The optode sensing agent of claim 1, wherein the sensor is soluble in an organic solvent.

19. The optode sensing agent of claim 1, wherein the core has a diameter of less than or equal to about 100 nm.

20. The optode sensing agent of claim 1, wherein the semipermeable membrane has a thickness of about 50 nm.

21. The optode sensing agent of claim 1, wherein the core further comprises a plasticizer.

22. A method of making oblong optode sensing agents, the method comprising:
a) providing a mold comprising one or more pores;
b) coating the interior surfaces of the one or more pores with a first material, the first material forming a semipermeable membrane;
c) applying a second material to the one or more coated pores from b) such that the second material fills the one or more coated pores, the second material comprising one or more sensors;
d) applying the first material to the ends of the one or more coated pores filled with the second material;
e) permitting the first and second materials to form the oblong optode sensing agents; and
f) releasing the oblong optode sensing agents from the mold.

23. The method of claim 22, wherein the first material is a biocompatible hydrogel.

24. The method of claim 23, wherein the first material is poly(2-hydroxyethyl methacrylate).

25. The method of claim 22, wherein the second material comprises a polymer.

26. The method of claim 25, wherein the polymer is selected from the group consisting of polyvinyl chloride, polylactic co-glycolic acid, methacrylate, and polycaprolactone

27. The method of claim 25, wherein the second material further comprises a plasticizer.

28. The method of claim 22, wherein the one or more pores have a width of about 200 nm.

29. The method of claim 22, wherein the mold is an aluminum oxide scaffold.

30. The method of claim 22, wherein releasing the oblong optode sensing agents from the mold further comprises etching the mold with an acid or a base

31. The method of claim 30, wherein releasing the oblong optode sensing agents further comprises sonication of the oblong optode sensing agents.

32. The method of claim 22, wherein the one or more sensors are fluorescent sensors.

33. A method of detecting an analyte in a tissue of a subject, the method comprising:
a) implanting a plurality of oblong optode sensing agents in the tissue, each oblong sensing agent comprising:
i) a core having one or more fluorescent sensors configured to bind to the analyte, and ii) a semipermeable membrane;
b) contacting the plurality of oblong sensing agents with the analyte;
c) detecting the analyte in the tissue.

34. The method of claim 33, wherein the analyte is selected from the group consisting of electrolytes, salts, hormones, steroids, small molecules, drugs, and saccharides.

35. The method of claim 33, wherein the core further comprises a polymer.

36. The method of claim 33, wherein the semipermeable membrane comprises a hydrogel.

37. The method of claim 36, wherein the semipermeable membrane comprises a biocompatible hydrogel.

38. The method of claim 37, wherein the semipermeable membrane is permeable to the analyte and impermeable to the one or more fluorescent sensors.

39. The method of claim 35, wherein the core comprises one or more polymers selected from the group consisting of polyvinyl chloride, polylactic co-glycolic acid, methacrylate, and polycaprolactone

40. The method of claim 33, wherein the plurality of oblong optode sensing agents has a circular cross-section.

41. The method of claim 33, wherein the plurality of oblong optode sensing agents has a rectangular shape.

42. The method of claim 40, wherein the plurality of oblong optode sensing agents has a length of about 40 μm to about 60 μm.

43. The method of claim 42, wherein the plurality of oblong optode sensing agents has a diameter of from about 200 nm to about 500 nm.

44. The method of claim 41, wherein the plurality of oblong optode sensing agents has a length of about 40 μm to about 60 μm.

45. The method of claim 44, wherein the plurality of optode sensing agents has a width of from about 200 nm to about 500 nm.

46. The method of claim 33, wherein detecting the analyte comprises (i) exciting the one or more fluorescent sensors in the plurality of oblong optode sensing agents with an excitation energy emission from an energy emission device and (ii) detecting fluorescent energy emitted by the one or more fluorescent sensors in the plurality of oblong optode sensing agents.

47. The method of claim 46, wherein the energy emission device is a handheld device.

48. The method of claim 37, wherein the semipermeable membrane comprises poly(2-hydroxyethyl methacrylate).

49. The method of claim 33, wherein the core has a diameter of less than or equal to about 100 nm.

50. The method of claim 33, wherein the semipermeable membrane has a thickness of about 50 nm.

51. The method of claim 33, wherein the core further comprises a plasticizer.

52. The method of claim 33, wherein implanting a plurality of oblong optode sensing agents comprises injecting the plurality of oblong optode sensing agents into the tissue.

53. The method of claim 33, wherein the tissue is selected from the group consisting of epidermal, muscular, ocular, endothelial, mucosal, dermal, subcutaneous, and organ tissues.
